(19) United States
(12) Patent Application Publication
(10) Pub. No.: US 2021/0338888 A1
(43) Pub. Date: Nov. 4, 2021

(54) ANTIMICROBIAL IMPLANT COATING

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: John Rose, Collierville, TN (US)

(21) Appl. No.: 17/243,084

(22) Filed: Apr. 28, 2021

Related U.S. Application Data
(60) Provisional application No. 63/017,831, filed on Apr. 30, 2020.

(51) Int. Cl. A61L 27/28; A61L 27/54; A61L 27/04; A61L 31/08; A61L 31/16; A01N 47/44

(52) U.S. Cl. CPC A61L 27/28; A61L 27/54; A61L 27/04; A61L 2300/206; A61L 31/16; A01N 47/44; A61L 2300/404; A61L 31/08

(57) ABSTRACT

Orthopedic implants having antimicrobial properties and methods of producing such orthopedic implants are provided. In various embodiments, the present disclosure pertains to orthopedic implants that comprise a metal substrate and a surface coating comprising chlorhexidine or a salt of chlorhexidine on a surface of the metal substrate. In various embodiments, the present disclosure also pertains to methods of making orthopedic implants that comprise: (a) pre-treating a metal substrate to form a pretreated metal substrate and (b) applying a coating comprising chlorhexidine or a salt of chlorhexidine on a surface of the pretreated metal substrate.

### CROSS-REFERENCE TO RELATED APPLICATIONS

This is a non-provisional of, and claims the benefit of the filing date of, pending U.S. provisional patent application No. 63/017,831, filed Apr. 30, 2020, entitled “ANTIMICROBIAL IMPLANT COATING” the entirety of which application is incorporated by reference herein.

### FIELD OF THE DISCLOSURE

The present disclosure relates generally to orthopedic implants and methods of producing the same. In particular, orthopedic implants having antimicrobial properties and methods of producing the same are provided.

### BACKGROUND OF THE DISCLOSURE

Implant related infections, while rare, are complications that usually require additional treatment and surgeries to resolve. In some cases, this can involve removal and replacement of the implant. The reason that removal and replacement may be required is that, when there is an infection, it is assumed that there is a biofilm on the implant. Biofilms cannot be removed by giving antibiotics to the affected patient.

Therefore, it would be beneficial to provide an improvement that reduces infection possibilities by applying a coating to implants that resists microbe attachment to implants during surgical procedures in a repeatable and cost-effective manner. It is with this in mind that the present disclosure is provided.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In various embodiments, the present disclosure pertains to orthopedic implants that comprise a metal substrate and a surface coating comprising chlorhexidine or a salt of chlorhexidine on a surface of the metal substrate.

In some embodiments, the surface coating is a substantially pure coating of chlorhexidine or a salt of chlorhexidine. As used herein a “substantially pure coating of chlorhexidine or a salt of chlorhexidine” is coating that contains 95 wt % or more, in some embodiments 97.5 wt % or more, in some embodiments 99 wt % or more, in some embodiments 99.5 wt % or more, 99.7 wt % or more, or even 99.9 wt % or more of chlorhexidine or a salt of chlorhexidine.

In some embodiments, the surface coating further comprises a hydrophilic polymer (e.g., the surface coating may comprise one, two or more hydrophilic polymers) and, optionally, a biodegradable polyester. In these embodiments, the amount of the chlorhexidine or salt of chlorhexidine may range, for example, from 10 wt % or less to nearly 100 wt %, for example, ranging anywhere from 10 wt % to 15 wt % to 20 wt % to 25 wt % to 30 wt % to 40 wt % to 50 wt % to 60 wt % to 70 wt % to 80 wt % to 90 wt % to 95 wt % to 97.5 wt % to 99 wt % (in other words, ranging between any two of the preceding values) and the amount of the hydrophilic polymer(s) and optional biodegradable polyester may range, for example, from 0 wt % to 90 wt %, for example, ranging anywhere from 0 wt % to 2.5 wt % to 5 wt % to 10 wt % to 15 wt % to 20 wt % to 25 wt % to 30 wt % to 40 wt % to 50 wt % to 60 wt % to 70 wt % to 80 wt % to 90 wt %.

Hydrophilic polymers may be selected from homopolymers and copolymers of alkylene oxides (e.g., homopolymers and copolymers of ethylene oxide, propylene oxide, etc.), including polyethylene glycol (PEG) (a poly(ethylene oxide) homopolymer) and poly(ethylene oxide)-poly(propylene oxide) block copolymers such as Pluronic F-127 (a poly(ethylene oxide)-poly(propylene oxide)-poly(ethylene oxide) triblock copolymer), modified celluloses (e.g., carboxymethyl cellulose (CMC), hydroxypropyl cellulose (HPC), hydroxypropyl methylcellulose (HPMC), etc.), polyvinyl alcohol, and biodegradable polyurethanes, among other hydrophilic polymers. Biodegradable polyesters may be selected, for example, from polylactides, polyglycolides, polylactide-polyglycolide blends, and copolymers containing lactide and glycolide (e.g., poly(lactide-co-glycolide)), among others.

In some embodiments, which may be used with any of the above embodiments, the orthopedic implant further comprises an additional coating comprising a hydrophilic polymer and, optionally, a biodegradable polyester, disposed over the surface coating, wherein the additional coating is different in composition from the surface coating. In some of these embodiments, the additional coating comprises chlorhexidine or a salt of chlorhexidine. In some of these embodiments, the additional coating does not comprise chlorhexidine or a salt of chlorhexidine. The hydrophilic polymer and the optional biodegradable polyester may be selected from those described above, among others.

In some embodiments, which may be used with any of the above embodiments, the orthopedic implant may be selected, for example, from an orthopedic joint implant, such as a joint implant for a hip (e.g., an acetabular shell, cup, cage, a femoral component, etc.), a knee (e.g., a femoral implant, a tibial implant, etc.), shoulder, wrist, interphalangeal, partial or carpal joint, ankle, elbow or the like; a spinal implant, such as a spinal spacer; an intramedullary rod; an intramedullary stem; an orthopedic nail, such as an intramedullary nail, femoral nail, tibial nail or the like; an orthopedic bone plate, such as a dynamic compression plate, locking compression plate or the like; or a fastener, such as a screw, wire, pin, or rod.

In some embodiments, which may be used with any of the above embodiments, the metal substrate may be formed from a number biocompatible metals including stainless steels, titanium, titanium alloys, including titanium-nickel alloys, cobalt chromium alloys, zirconium, oxidized zirconium, zirconium alloys, niobium, niobium alloys, hafnium, hafnium alloys, tantalum, tantalum alloys, and nickel-chromium alloys, among others.

In some embodiments, which may be used with any of the above embodiments, the salt of chlorhexidine may be selected, for example, from chlorhexidine digluconate, chlorhexidine diacetate, chlorhexidine dihydrochloride, chlorhexidine phosphanilate or chlorhexidine dinalidixate.

In some embodiments, which may be used with any of the above embodiments, the chlorhexidine or salt of chlorhexidine may be present on the metal substrate in an amount ranging from 0.025 μg/cm² or less to 1000 μg/cm² or more (e.g. ranging anywhere from 0.025 to 0.5 to 1 to 2.5 to 5 to 10 to 25 to 50 to 100 to 250 to 500 to 1000 μg/cm²).

Where the metal substrate comprises a porous region (e.g., a porous bone ingrowth region), the chlorhexidine or salt of chlorhexidine may be present on the metal substrate in an amount based on the void volume of the porous region (e.g., expressed in units of μg chlorhexidine or salt of chlorhexidine per cm³ void volume). In some these embodiments, which may be used with any of the above embodiments, the amount of the chlorhexidine or salt of chlorhexidine may range from 10 μg/cm³ or less to 5000 μg/cm³ or more (e.g., ranging anywhere from 10 μg/cm³ to 25 μg/cm³ to 50 μg/cm³ to 100 μg/cm³ to 250 μg/cm³ to 500 μg/cm³ to 1000 μg/cm³ to 2500 μg/cm³ to 5000 μg/cm³).

In some embodiments, which may be used with any of the above embodiments, the orthopedic implant is an intramedullary nail and the chlorhexidine or salt of chlorhexidine is present on the metal substrate in a surface concentration ranging from 150 to 300 μg/cm².

In some embodiments, which may be used with any of the above embodiments, the orthopedic implant is a knee or hip implant and the chlorhexidine or salt of chlorhexidine is present on the metal substrate in a surface concentration ranging from 0.1 to 100 ug/cm² For some implants, there may be different concentrations of chlorhexidine or salt of chlorhexidine in different areas. For example, for a hip implant, a low concentration of chlorhexidine or salt of chlorhexidine may be provided on the ingrowth surface of a hip stem, a higher concentration of chlorhexidine or salt of chlorhexidine be provided on the neck of the hip stem, and very little to no chlorhexidine or salt of chlorhexidine may be provided on the articulating surfaces.

In various embodiments, which may be used with any of the above embodiments, the orthopedic implant may be made by a process that comprises pre-treating the metal substrate with an acidic or basic solution to form a pretreated metal substrate, prior to applying the surface coating.

In various embodiments, which may be used with any of the above embodiments, the orthopedic implant may be made by a process that comprises pre-treating the metal substrate with a solution of phosphoric acid or a salt thereof to form a pretreated metal substrate, prior to applying the surface coating. Salts of phosphoric acid include alkali-metal (e.g., sodium, potassium, lithium, rubidium, cesium, and francium) salts of phosphoric acid and include mono-alkali-metal phosphate salt solutions (e.g., monosodium phosphate salt solution, monopotassium phosphate salt solution, monolithium phosphate salt solution, etc.), di-alkali-metal phosphate salt solutions (e.g., disodium phosphate salt solution, dipotassium phosphate salt solution, dilithium phosphate salt solution, etc.).

In various embodiments, which may be used with any of the above embodiments, the surface coating may be formed on the pretreated metal substrate by contacting the pretreated metal substrate with a surface coating solution comprising the chlorhexidine or a salt of chlorhexidine (and, in some embodiments, the hydrophilic polymer and optional biodegradable polyester).

In various embodiments, which may be used with any of the above embodiments, the surface coating may be applied to the pretreated metal substrate by dip coating the pretreated metal substrate in the surface coating solution.

In various embodiments, which may be used with any of the above embodiments, the surface coating may be applied to the pretreated metal substrate by a process that comprises (i) at least partially immersing the metal substrate in the surface coating solution and (ii) withdrawing the pretreated metal substrate from the surface coating solution to form a deposited layer. In certain of these embodiments, the pretreated metal substrate is withdrawn from the surface coating solution at a rate ranging, for example, from 20 to 1000 mm/minute. The ultimate speed that is selected may be dependent on a range of variables including, for example, the concentration of the chlorhexidine or chlorhexidine salt in the surface coating solution, and the roughness of the metal surface.

In various embodiments, which may be used with any of the above embodiments, the additional coating may be formed on the metal substrate with the surface coating by contacting the metal substrate with the surface coating with an additional coating solution comprising the hydrophilic polymer and optional biodegradable polyester.

In various embodiments, which may be used with any of the above embodiments, the additional coating may be applied to the metal substrate with the surface coating by dip coating the metal substrate with the surface coating in the additional coating solution.

In various embodiments, which may be used with any of the above embodiments, the additional coating may be applied to the metal substrate with the surface coating by a process that comprises (i) at least partially immersing metal substrate with the surface coating in the additional coating solution and (ii) withdrawing the metal substrate with the surface coating from the additional coating solution to form a deposited layer. In certain of these embodiments, the metal substrate with the surface coating is withdrawn from the additional coating solution at a rate ranging, for example, from 20 to 1000 mm/minute. The ultimate speed that is selected may be dependent on a range of variables including, for example, the concentration of the hydrophilic polymer and optional biodegradable polyester in the additional coating solution.

In various embodiments, which may be used with any of the above embodiments, the surface coating solution may comprise between 1 and 80 wt. percent of the chlorhexidine or salt of chlorhexidine.

In various embodiments, which may be used with any of the above embodiments, the surface coating solution may be an aqueous solution.

In various embodiments, which may be used with any of the above embodiments, the surface coating solution may comprise an organic solvent. For example, the surface coating solution may comprise an organic solvent containing one or more of the following solvents, among others: polar organic solvents, protic organic solvents, aprotic organic solvents, aliphatic organic solvents, or aromatic organic solvents, with dichloromethane, N-methyl-2-pyrrolidone (NMP), methanol, ethanol, isopropanol, acetonitrile, acetone, tetrahydrofuran, methylene chloride, toluene, methyl ethyl ketone, or dimethyl sulfoxide being specific examples of organic solvents.

In various embodiments, which may be used with any of the above embodiments, the additional coating solution may comprise between 1 and 80 wt. percent of the hydrophilic polymer and optional biodegradable polyester.

In various embodiments, which may be used with any of the above embodiments, the additional coating solution may be an aqueous solution.

In various embodiments, which may be used with any of the above embodiments, the additional coating solution may comprise an organic solvent. For example, the additional coating solution may comprise an organic solvent containing one or more of the following solvents, among others: polar organic solvents, protic organic solvents, aprotic organic solvents, aliphatic organic solvents, or aromatic organic solvents, with dichloromethane, N-methyl-2-pyrrolidone (NMP), methanol, ethanol, isopropanol, acetonitrile, acetone, tetrahydrofuran, methylene chloride, toluene, methyl ethyl ketone, or dimethyl sulfoxide being specific examples of organic solvents.

In various embodiments, the present disclosure pertains to methods of making orthopedic implants that comprise: (a) pre-treating a metal substrate to form a pretreated metal substrate and (b) applying a surface coating comprising chlorhexidine or a salt of chlorhexidine on a surface of the pretreated metal substrate. In some embodiments, the methods of making orthopedic implants further comprise: (c) applying an additional coating comprising a hydrophilic polymer and optional biodegradable polyester over the surface coating. In some embodiments, the orthopedic implant that is made may be selected from those set forth above, among others. In some embodiments, the metal substrate may be formed from a number biocompatible metals including those set forth above, among others. In some embodiments, the salt of chlorhexidine that is used may be selected from those set forth above, among others. In some embodiments, the chlorhexidine or salt of chlorhexidine may be present in a surface concentration range selected from those set forth above, among others. In some embodiments, the hydrophilic polymer and optional biodegradable polyester may be selected from those set forth above, among others.

In various embodiments, which may be used with any of the above embodiments, the methods comprise pre-treating the metal substrate with an acidic or basic solution to form the pretreated metal substrate.

In various embodiments, which may be used with any of the above embodiments, the methods comprise pre-treating the metal substrate with a solution of phosphoric acid or a salt thereof to form the pretreated metal substrate. Salts of phosphoric acid include those set for above, among others.

In various embodiments, which may be used with any of the above embodiments, the surface coating may be formed on the pretreated metal substrate by contacting the pretreated metal substrate with a surface coating solution comprising chlorhexidine or a salt of chlorhexidine and, in some embodiments, a hydrophilic polymer and optional biodegradable polyester. In certain embodiments, the surface coating may be applied to the pretreated metal substrate by dip coating the pretreated metal substrate in the surface coating solution. In certain embodiments, the surface coating may be applied to the pretreated metal substrate by a process that comprises (i) at least partially immersing the metal substrate in the surface coating solution and (ii) withdrawing the pretreated metal substrate from the surface coating solution to form a deposited layer. For example, the pretreated metal substrate may be withdrawn from the surface coating solution at a rate ranging from 20 to 1000 mm/minute, among other values.

In various embodiments, which may be used with any of the above embodiments, the additional coating may be applied to the metal substrate and surface coating by contacting the metal substrate with the surface coating with an additional coating solution that comprises a hydrophilic polymer and optional biodegradable polyester. In certain embodiments, the additional coating may be applied to the metal substrate with the surface coating by dip coating the metal substrate with the surface coating in the additional coating solution. In certain embodiments, the additional coating may be applied to the metal substrate with the surface coating by a process that comprises (i) at least partially immersing the metal substrate with surface coating in the additional coating solution and (ii) withdrawing the metal substrate with surface coating from the additional coating solution to form a deposited layer. For example, the metal substrate with the surface coating may be withdrawn from the additional coating solution at a rate ranging from 20 to 1000 mm/minute, among other values.

In various embodiments, which may be used with any of the above embodiments, the surface coating solution used in the methods described herein may be an aqueous solution.

In various embodiments, which may be used with any of the above embodiments, the surface coating solution used in the methods described herein may comprise an organic solvent, which may be selected, for example, from those set forth above, among others.

In various embodiments, which may be used with any of the above embodiments, the additional coating solution used in the methods described herein may be an aqueous solution.

In various embodiments, which may be used with any of the above embodiments, the additional coating solution used in the methods described herein may comprise an organic solvent, which may be selected, for example, from those set forth above, among others.

In various embodiments, which may be used with any of the above embodiments, the metal substrate used in the orthopedic implants and methods described herein may have a textured surface. For example, a textured surface may be formed by a variety of surface texturing methods, including chemical etching, electro-etching, anodizing, grit blasting, and cold spray coating processes among others.

Further features and advantages of at least some of the embodiments of the present disclosure, as well as the operation of various embodiments of the present disclosure, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 shows a scanning electron micrograph of a chlorhexidine digluconate coating applied to a titanium alloy pin without the acid or base pre-treatment;

FIG. 2 shows a scanning electron micrograph of a chlorhexidine digluconate coating applied to a titanium alloy pin after pre-treatment with phosphoric acid solution;

FIG. 3 shows a scanning electron micrograph of a chlorhexidine digluconate coating applied to a titanium alloy pin after pre-treatment with a basic disodium phosphate solution;

FIG. 4 shows a scanning electron micrograph of a chlorhexidine digluconate coating applied to a stainless steel pin after pre-treatment with a basic disodium phosphate solution;

FIG. 5 shows a scanning electron micrograph of a chlorhexidine digluconate coating applied to a stainless steel pin after pre-treatment with phosphoric acid solution;

FIG. 6 shows counts for S. Aureus after incubation with different concentrations of chlorhexidine digluconate.

FIG. 7 shows counts for P. aeruginosa after incubation with different concentrations of chlorhexidine digluconate.

FIG. 8 is a photograph of the effect of an IM nail coated with chlorhexidine digluconate (CHG) that was touched with a wet glove.

FIG. 9 shows a wipe test setup.

FIG. 10 is a SEM image of Pin M-5-6-20-13 (coated with CHG+PEG/CHG #4) that represents a batch of pins sent for testing.

FIG. 11 is a SEM image of pin M-6-1-20-13 (coated with CHG+CHG/F127 #2) that represents a batch of pins sent for testing.

FIG. 12 is a SEM image of pin M-6-8-20-13 (coated with CHG+1% CMC) that represents a batch of pins sent for testing.

FIG. 13 is a SEM image of pin M-6-11-20-13 (coated with CHG+0.75% CMC) that represents a batch of pins sent for testing.

FIG. 14 is a bar graph illustrating the average percentage of coating removed from pins coated with overcoats (see Table 3 for coating formulations).

FIG. 15 shows microbe counts for the surface for the control and titanium pins coated with overcoats.

FIG. 16 shows microbe counts for the solution for the control and titanium pins coated with overcoats.

FIG. 17 is a photograph of the aftermath of the wet glove test performed on a nail coated with CHG (M-6-18-20-1).

FIG. 18 is a photograph of the aftermath of the wet glove test performed on a nail coated with the overcoat CHG+PEG/CHG #4 (M-6-18-20-2).

FIG. 19 is a photograph of the aftermath of the wet glove test performed on a nail coated with the overcoat CHG+1% CMC (M-6-18-20-3).

FIG. 20 is a photograph of the aftermath of the wet glove test performed on a nail coated with the overcoat CHG+0.75% CMC (M-6-22-20-4).

FIG. 21 is a photograph of the aftermath of the wet glove test performed on a nail coated with the overcoat CHG+PEG/F127 #2 (M-6-22-20-5).

FIG. 22 is a SEM image of Pin M-5-4-20-13 (coated with PP-1) that represents a batch of pins sent for testing.

FIG. 23 is a SEM image of pin M-6-2-20-13 (coated with PP-2) that represents a batch of pins sent for testing.

FIG. 24 is a SEM image of pin M-6-29-20-13 (coated with PP-5) that represents a batch of pins sent for testing.

FIG. 25 is a SEM image of pin M-8-4-20-13 (coated with PEG/CHG #9) that represents a batch of pins sent for testing.

FIG. 26 is a bar graph illustrating the average percentage of coating removed from pins coated with blends (see Table 4 for coating formulations).

FIG. 27 shows microbe counts for the surface for the control and titanium pins coated with blends.

FIG. 28 shows microbe counts for the solution for the control and titanium pins coated with blends

FIG. 29 is a photograph of the aftermath of the wet glove test performed on a nail coated with CHG (M-6-18-20-1).

FIG. 30 is a photograph of the aftermath of the wet glove test performed on a nail coated with the blend PP-1 (M-6-22-20-6).

FIG. 31 is a photograph of the aftermath of the wet glove test performed on a nail coated with the blend PP-2 (M-6-22-20-7).

FIG. 32 is a photograph of the aftermath of the wet glove test performed on a nail coated with the blend PEG/CHG #4 #2 (M-7-13-20-8).

FIG. 33 is a photograph of the aftermath of the wet glove test performed on a nail coated with the blend PP-5 (M-7-13-20-9).

FIG. 34 is a photograph of the aftermath of the wet glove test performed on a nail coated with the blend PEG/CHG #9 (M-8-3-20-10).

### DETAILED DESCRIPTION

Embodiments of the present will now be described more fully hereinafter with reference to the accompanying drawings, in which preferred embodiments of the present disclosure are presented.

In some embodiments, a goal of the present disclosure is to reduce orthopedic implant related infections by treating implants so that they are less likely to become colonized by bacteria during use. The treatment is the formation of a coating that is able to resist bacterial colonization after implantation of the orthopedic implant. The coatings described herein utilize a chlorhexidine compound as the antimicrobial agent. After implantation, the chlorhexidine compound is released from the implant surface killing bacteria in the local environment. Removing bacteria in the local environment around the implant site helps prevent bacterial colonization of the implant.

It has found that it is possible to coat metal substrate surfaces such as titanium alloys and stainless steel, among others, with a chlorhexidine compound which will later release when implanted. It has also been found that pre-treatment of the metal substrate surfaces some cases allows a uniform coating to be achieved. Without pre-treatment, the coating was found to be patchy and inconsistent in thickness and coverage in some cases.

In particular, substantially pure chlorhexidine digluconate (CHG) coatings demonstrated promising results for use on both titanium and stainless steel. Using such coatings, it was possible to prevent colonization for 2 days for S. aureus and 1 day for P. aeruginosa. Microbiology results were best for titanium alloys when they were pre-treated with phosphoric acid. Testing of chlorhexidine digluconate coatings on stainless steel was also successful. A pre-treatment with a dibasic sodium phosphate dibasic solution led to greater success on stainless steel.

Specific examples are described below.

### Example 1

A titanium 6Al 4V alloy rod was machined into a test pin 2 mm in diameter and 85 mm long. A first group of pins were ultrasonically cleaned first in a basic cleaning solution (Micro-90® from Cole-Parmer, Vernon Hills, Ill., USA) and then in DI water. The pins were then dried prior to coating.

In a second group of pins, a pre-treatment consisting of being immersed in a 0.5M phosphoric acid solution for 5 minutes, followed by rinsing in DI water, was carried out. Pins were coated immediately, or coated after drying in a 50° C. oven for 30 minutes, or coated after drying in a 50° C. oven for 1 hour.

In a third group of pins, a pre-treatment consisting of being immersed in a 0.5 M dibasic sodium phosphate solution for 5 minutes was carried out. Pins were coated immediately, or coated after drying in a 50° C. oven for 30 minutes, or coated after drying in a 50° C. oven for 1 hour.

Pin coating was carried out by dipping the pins into a 20 wt/vol % chlorhexidine digluconate solution at a speed of 300 mm/min and after a 5 second dwell, removed at the same speed. After coating pins were dried in a 50° C. oven for 1 hour, the amount of coating was evaluated by weighing the pin before and after coating. The coating density was calculated based on the weight before and after coating, the measured length of coating (l), and assuming a nominal 1 mm pin radius (r), specifically, (final weight-starting weight)/coated surface area, where coated surface area is 2πrl+2πr².

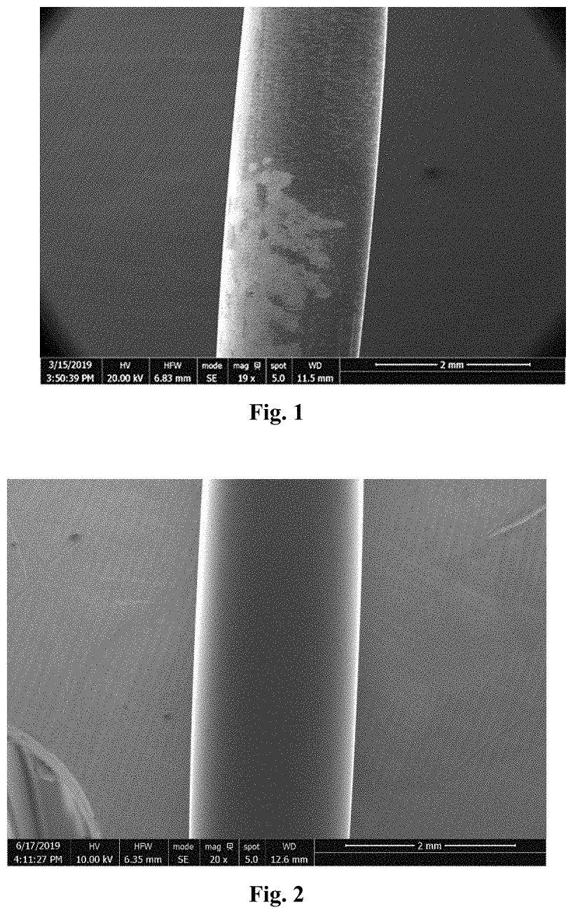

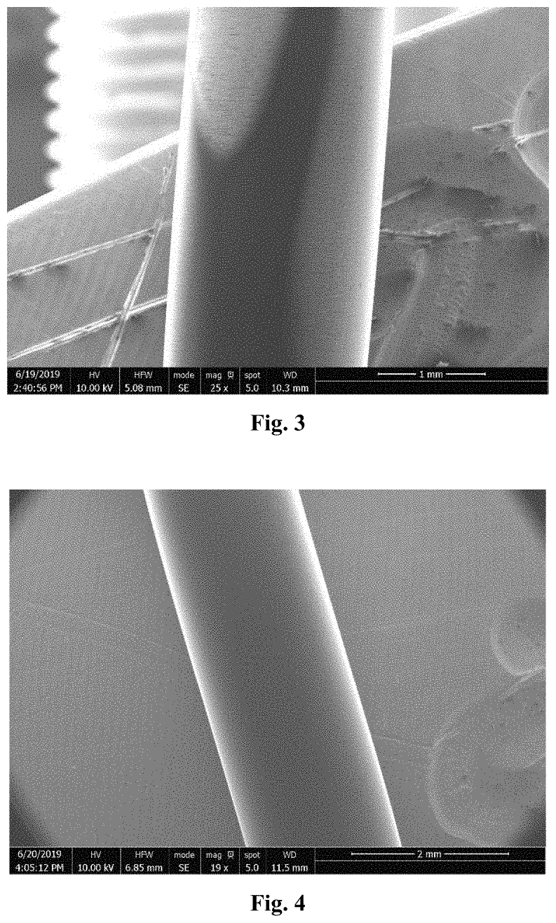

After drying the pins were examined in a scanning electron microscope to evaluate the coatings. The pins coated without the acid pre-treatment had an inconsistent coating (see FIG. 1). The pins coated with the acid pre-treatment were seen to be uniform, see (FIG. 2, representing drying time of 60 minutes) with coating rates ranging from 218-242 μg/cm². Similar results were obtained, whether pins were pre-treated in phosphoric acid 0.5 minute, 1 minute, 2.5 minutes, or 5 minutes. The pins coated with the base-pretreatment had a less uniform coating (see FIG. 3, representing drying time of 0 minutes) with coating rates ranging from 156-164 μg/cm².

### Example 2

Stainless steel 316 alloy rod was machined into a test pin 2 mm in diameter and 85 mm long. In a first group, pins were ultrasonically cleaned first in a basic cleaning solution and then in DI water. The pins were then dried prior to coating.

In a second group of pins, a pre-treatment consisting of being immersed in a dibasic sodium phosphate dibasic solution for 5 minutes was carried out. Pins were coated immediately, or coated after drying in a 50° C. oven for 30 minutes, or coated after drying in a 50° C. oven for 1 hour.

In a third group of pins, a pre-treatment consisting of being immersed in a 0.5M phosphoric acid solution for 5 minutes, followed by rinsing in DI water, was carried out. Pins were coated immediately, or coated after drying in a 50° C. oven for 30 minutes, or coated after drying in a 50° C. oven for 1 hour.

Pin coating was carried out by dipping the pins into a 20 wt/vol % chlorhexidine digluconate solution at a speed of 300 mm/min and after a 5 second dwell, removed at the same speed. After coating, pins were dried in a 50° C. oven for 1 hour. The amount of coating was evaluated by weighing the pin before and after coating. The coating density was calculated based on the weight before and after coating, the measured length of coating (l), and assuming a nominal 1 mm pin radius (r), specifically, (final weight-starting weight)/coated surface area, where coated surface area is 2πr+2πr².

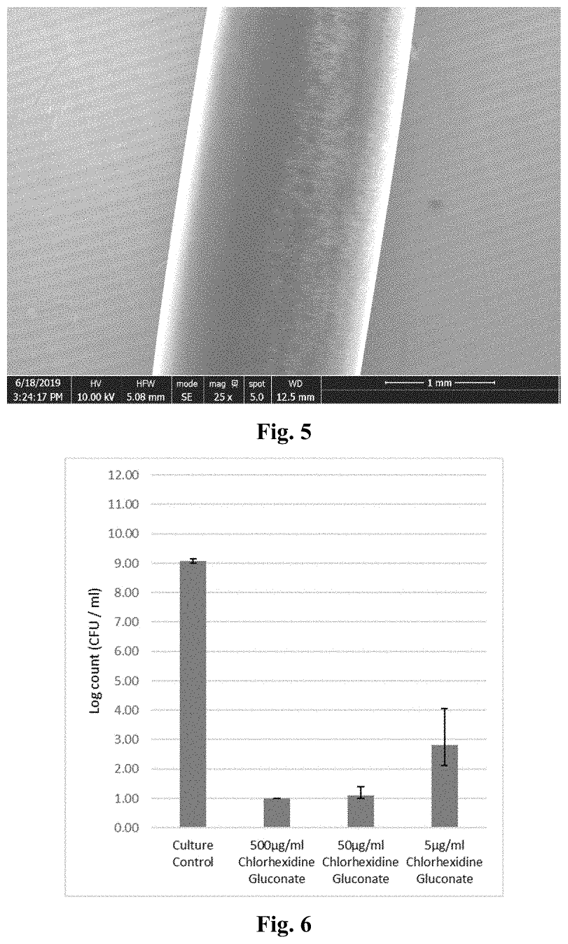

After drying the pins were examined in a scanning electron microscope to evaluate the coatings. The pins coated without the acid or base pre-treatment had an inconsistent coating (not shown). The pins coated with the base pre-treatment were seen to be more uniform see (FIG. 4, representing drying time of 0 minutes) with coating rates ranging from 197-259 μg/cm². The pins coated with the acid-pretreatment were seen to have a less uniform coating (see FIG. 5, representing drying time of 60 minutes) with coating rates ranging from 223-240 μg/cm².

### Example 3

Titanium alloy samples were coated with chlorhexidine after acid treatment as described in Example 1. The pins were then gamma sterilized and submitted for microbiology testing. In this microbiology test the samples are placed in a 100% serum containing 5×10⁴ CFU of a targeted microorganism for 24 hours. After this time, they are removed, rinsed and placed in DI water. They are then sonicated and vortexed. This solution is then diluted and plated onto agar culture plates. After a 24-hour incubation the number of colonies is counted.

When tested against S. aureus and P. aeruginosa the pins coated with chlorhexidine digluconate were found to have no bacteria adhered to the surface. Uncoated control samples had approximately 10⁴ CFU of microbes adhered to their surface.

### Example 4

316 Stainless steel pins were first treated with dibasic sodium phosphate and then coated with chlorhexidine digluconate as described in Example 2. After gamma sterilization they were testing using the method described in Example 3 against S. aureus. The results showed no bacteria adhered to the surface, whereas an uncoated control had approximately 10⁴ CFU of microbes adhered to the surface.

### Example 5

Testing was done by spiking chlorhexidine digluconate into a suspension of 10 CFU/ml of either S. aureus or P. aeruginosa in 100% bovine serum. Samples were incubated at 37° C. for 24 hours, after which the concentration of microbes remaining in the suspension were measured by plating and counting.

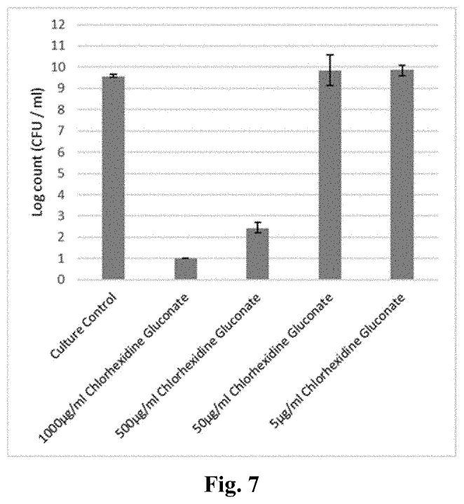

The results for S. aureus. aeruginosa are shown in FIG. 6. The results for P. aeruginosa are shown in FIG. 7.

From these data, the desired minimum surface concentrations of chlorhexidine digluconate for an intramedullary nail (IM nail) was estimated. The table below, gives these minimum surface concentrations for the straight section of an IM Nail. These values assume 2 mm over-ream for the IM nail, and that the IM nail requires to have enough chlorhexidine digluconate to reach the MBC (minimal bactericidal concentration) for P. aeruginosa.

| Nail shaft | Surface area | Volume outside | Surface concentration of chlorohexidine |
|---|---|---|---|
| diameter | of 1 cm length | nail with 2 mm | to reach 1000 ug/cm³ in volume outside |
| mm | on nail cm² | overream cm³ | IM nail ug/cm² |
| 8.5 | 2.67 | 0.66 | 247 |
| 10 | 3.14 | 0.75 | 240 |
| 11.5 | 3.61 | 0.85 | 235 |
| 13 | 4.08 | 0.94 | 231 |

Desired surface concentrations of chlorhexidine digluconate for a hip implant was also estimated. For this estimate, calculation of the required surface concentration of an antimicrobial for a beaded surface is based on a desired elution concentration within the beaded surface of 1000 μg/cm³.

Assumptions are as follows: bead diameter is 0.01 inch (0.0254 cm); bead radius is 0.005 inch (0.0127 cm); the coat is press fitted into bone, i.e., no free space above porous layer (this case requires the minimum amount of coating on the beads); best packing of spheres is based on the face centered cubic (FCC) model (also equivalent to hexagonal close packing); the packing fraction of an FCC unit cell is 0.74; there are 4 spheres in a FCC unit cell; the side of a FCC unit cell is 2 √(2)*r, where r is the radius of the sphere.

Based on the above, the following can be calculated: (a) Volume of unit cell=(2 √(2)*r)³=22.6 r³; (b) Free space in a unit cell=(1−0.74)*Volume of unit cell=22.6*0.26*r³=5.88 r³; (c) Surface area of a sphere=4πr² and Surface area of 4 spheres=4*4πr²=16πr²; (d) Surface coating concentration (in μg/cm²) to achieve 1000 μg/cm³ in free space of a FCC unit cell (assuming all coating elutes) is: 1000*5.88 r³/16πr²=5880 r³/16πr²; for a 0.0127 cm radius sphere, this corresponds to 5880*(0.0127)³/16*π*(0.0127)² μg/cm²=1.48 μg/cm².

### Example 6

As seen from the preceding Examples, substantially pure chlorhexidine digluconate coatings on both titanium and stainless steel demonstrate pin demonstrate good resistance against S. aureus and P. aeruginosa.

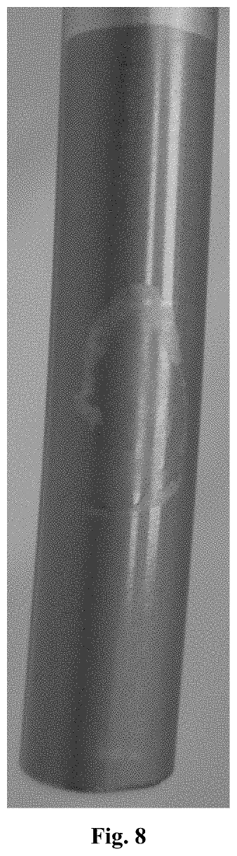

However, substantially pure chlorhexidine digluconate coatings do not have good glove resistance. FIG. 8 illustrates the effect of touching a wet glove to a nail coated with substantially pure chlorhexidine digluconate. The coating is completely removed down to the surface of the nail, as shown by the lighter area shaped as an incomplete circle in FIG. 8. Thus, for at least those portions of orthopedic implants that are subject to contact during use (e.g., with a glove), further coatings are desired.

In this example, various solutions and processes were experimented with to evaluate the glove resistance of coatings. Coatings were applied to titanium 6-4 pins for evaluations. Pins were 2 mm in diameter and 85 mm long with a break-off tab to enable the pins to be held during coating.

Prior to coating all pins were cleaned in a basic cleaning solution (Micro-90®) in an ultrasonic bath. All pins were weighed in triplicate before coating. If pins were dipped in an acid pre-treatment, they were placed in the solution for 5 minutes, then rinsed thoroughly in DI water. The pins dipped in acid treatment were dried in the 50° C. oven for 30 minutes, then cooled for 10 minutes at room temperature before being dip coated. The solution used for dipping was poured into a 50 ml polypropylene centrifuge tube to ensure that the depth of the solution was sufficient to coat a pin. Pins were then dip coated using the dip coater. For all dipping the coating solution, dip speed, and dwell time were recorded. The pins were dried in the 50° C. oven for 60 minutes after each dip, then cooled for 10 minutes at room temperature. After all dips were completed and the pin was dried appropriately, the length of the coating along the pin was measured with calipers. The pins were then weighed in triplicate again after coating. Using the length of coating, the weight before and after coating and assuming a nominal 2 mm pin diameter, the coating weight was calculated.

$$coating weight ( \frac{μg}{{cm}^{2}} ) = 1 \times {10}^{6} \times \frac{final weight ( g ) - starting weight ( g )}{coated surface area ( {cm}^{2} )}$$

$$coated surface area = 2 π ri + π {r}^{2}$$

$$where r ( cm ) is the radius of the pin and l ( cm ) is the length of the coated region$$

The pins were viewed using a Scanning Electron Microscope (SEM FEI Quanta 650) and Energy-Dispersive Analysis X-ray (EDAX Element) in some cases, and pictures were taken to evaluate the consistency of the coating.

Chemicals used in this work were:
Chlorhexidine digluconate solution, 20% in water, Alfa Asar, Haverhill, Mass., USA, cat #41385, lot #Q30E057
Chlorhexidine (pure CHG), 1 g vials, Sigma-Aldrich, St. Louis, Mo., USA, cat #PHR1294-1G, lot #LRACO252
Polymer (liquid PEG): Polyethylene glycol 3000 monodisperse solution, Sigma-Aldrich, lot #BCBS6457V
Polymer (PEG): Poly(ethylene glycol), Aldrich Chemistry, lot #BCBZ9502, cat #81300, average Mn 20,000
Sodium carboxymethyl cellulose, Na-CMC (CMC), Sigma, medium viscosity, cat #C-4888, lot #71H0397
CMC, Sigma, high viscosity, cat #C-5013, lot #32H0921
Pluronic F-127 (poly(ethylene oxide)-poly(propylene oxide)-poly(ethylene oxide) triblock copolymer), Sigma-Aldrich, cat #P2443-250G, batch #077K0020
Purasorb® PDL-20 (poly(D,L-lactide)), Corbion Purac, Amsterdam, Netherlands, lot #1508002112
L-cysteine hydrochloride, Sigma-Aldrich, lot #BCCB8587
L-Glutathione, Sigma-Aldrich, lot #SLCB5110
Hydroxypropyl cellulose (HPC), Sigma, cat #435007, lot #MRCK8238
Hydroxypropyl methylcellulose (HPMC), Sigma, cat #H3785, lot #SLCC8612
Dibasic sodium phosphate solution, 0.5 M in H₂O, Sigma-Aldrich, cat #94096, lot #BCBZ5133
Phosphoric acid, Baker Analyzed, J.T. Baker Inc., Phillipsburg, N.J., USA cat #02600-00, lot #G23818
Alginic Acid, Sigma-Aldrich, cat #180947-100g, lot #MKCJ1280
Solvent, Dichloromethane, Sigma-Aldrich, lot #SHBF7914V, cat #270997
DI water

The phosphoric acid was used to make a 0.5 Molar solution by pipetting 3.38 ml of phosphoric acid into 25 ml of DI water and then adding DI water to adjust the final volume to 100 ml.

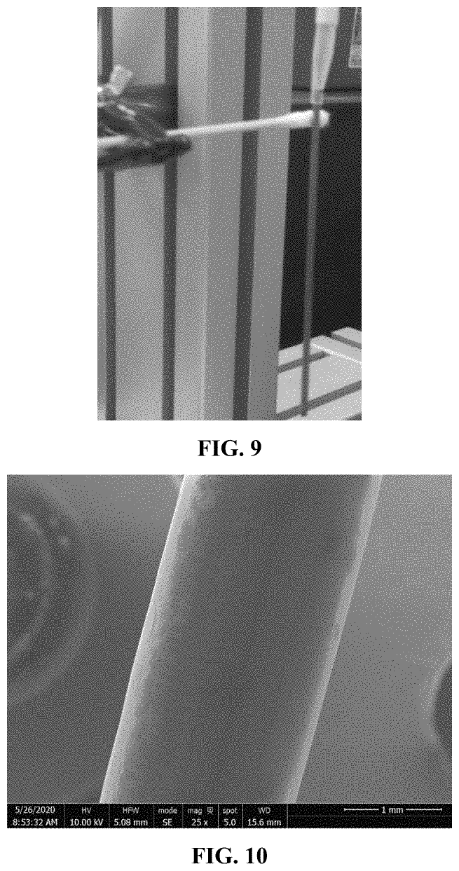

All wipe test methods were conducted after weighing the coated pin, and all methods used a dip coater at 500 mm/min to run a wet q-tip from the breakoff tab to the tip of the pin. The wipe test setup is shown in FIG. 9.

### CHG Controls

Pins were coated as CHG controls in order to compare the methods of wipe testing. The CHG control pins were dipped in acid treatment for 5 minutes, thoroughly rinsed in DI water, and left in the 50° C. oven to dry for 30 minutes. The pins were then dipped in CHG at 300 mm/min with a 5 second dwell time, and dried in the oven for 60 minutes. Table 1 displays the batch numbers of the pins coated as CHG controls, the coating weights before and after conducting the wipe test, and the percentage of coating removed from the wipe test. By completing testing of these CHG control pins on the various methods of wipe testing, it was determined that the methods are all comparable to each other since the percentage of coating removed on the CHG control remained approximately the same through all methods.

TABLE 1

| | Coating Weight | Coating Weight | |
|---|---|---|---|
| | before Wipe | after Wipe | % Coating |
| Batch Number | Test (μg/cm²) | Test (μg/cm²) | Removed |
| M-3-23-20-4 | 290 | 255 | 11.9 |
| M-3-23-20-5 | 160 | 146 | 9.1 |
| M-3-23-20-6 | 248 | 222 | 10.5 |
| M-4-6-20-16 | 269 | 244 | 9.1 |
| M-4-6-20-17 | 245 | 224 | 8.6 |
| M-4-6-20-18 | 250 | 227 | 9.8 |
| M-5-21-20-4 | 215 | 207 | 3.7 |
| M-5-21-20-5 | 197 | 180 | 8.6 |
| M-5-21-20-6 | 204 | 189 | 7.7 |
| M-6-22-20-8 | 227 | 203 | 10.7 |
| M-6-22-20-9 | 228 | 195 | 14.5 |
| M-6-22-20-10 | 213 | 177 | 17.0 |

### Test Solutions

Table 2 shows the solutions prepared and used for glove resistance testing.

TABLE 2

| Name of Solution | Components of Solution |
|---|---|
| PEG/CHG #3 | 20 mL CHG, 8 g PEG |
| PEG/CHG #4 | 20 mL CHG, 12 g PEG, 10 mL DI water |
| PEG/CHG | 20 mL CHG, 4 g PEG, 15 mL DI water |
| PVA | 5 g PVA, 200 mL DI water |
| 1% CMC | 2 g Na-CMC, 200 mL DI water |
| PP-1 | 1 g pure CHG, 1 g PDL-20, 1 g PEG, 15 mL NMP |
| PP-2 | 1 g pure CHG, 1 g PDL-20, 2 g PEG, 15 mL NMP |
| PP-3 | 5 mL CHG, 5 mL NMP, 1 g PDL-20, 1 g PEG |
| PP-4 | 1 g CHG, 15 mL solvent, 1 g PDL-20, 1 g PEG |
| 1% Alginic acid | 1 g alginic acid, 100 mL DI water |
| F127 10% | 10 g Pluronic F127, 100 mL DI water |
| AG1 | 2 mL 1% alginic acid, 18 mL DI water |
| AG2 | 2 mL AG1, 18 mL DI water |
| AG3 | 2 mL AG2, 18 mL DI water |
| FA | 10 mL F127 10%, 10 mL AG3 |
| FB | 10 mL F127 10%, 10 mL AG2 |
| PEG/F127 #1 | 20 mL CHG, 4 g Pluronic F127, 9 g PEG, 10 mL DI water |
| PEG/F127 #2 | 20 mL CHG, 3 g Pluronic F127, 10 mL DI water |
| PEG#3G | 20 mL CHG, 3 g PEG, 10 mL DI water |
| CMC #1 | 20 mL CHG, 0.2 g medium viscosity CMC |
| PEG/CHG #4d | 10 mL CHG, 6 g PEG, 20 mL DI water |
| CMC #2 | 20 mL CHG, 0.2 g medium viscosity CMC, 10 mL DI water |
| 0.1% CMC | 3 mL 1% CMC soln, 27 mL DI water |
| 0.01% CMC | 3 mL 0.1% CMC soln, 27 mL DI water |
| 0.5% CMC | 1 g Na-CMC, 200 mL DI water |
| CMC/CHG #3 | 10 μL CHG, 207 μg medium viscosity CMC, 20 mL DI water |
| CMC/CHG #4 | 10 μL CHG, 302 μg medium viscosity CMC, 30 mL DI water |
| CMC/CHG #5 | 150 μL CHG, 300 mg medium viscosity CMC, 30 mL DI water |
| CMC/CHG #6 | 75 μL CHG, 294 μg medium viscosity CMC, 30 mL DI water |
| L-cysteine | 30 mL L-cysteine, 300 mL DI water |
| hydrochloride, 1% in | |
| DI water | |
| L-Glutathione solution, | 30 mL L-Glutathione, 300 mL DI water |
| 1% in DI water | |
| CMC/CHG #7 | 112 μg CHG, 0.3 g medium viscosity CMC, 30 mL DI water |
| CHG/HPC #1 | 10 mL CHG, 0.3 g HPC, 20 mL DI water |
| CHG/HPMC #1 | 10 mL CHG, 0.3 g HPMC, 20 mL DI water |
| CMC/CHG #8 | 75 μL CHG, 0.4 g CMC, 30 mL DI water |
| CMC/CHG #9 | 75 μL CHG, 0.3 g high viscosity CMC, 30 mL DI water |
| 0.75% CMC | 0.225 g medium viscosity CMC, 30 mL DI water |
| PEG/CHG #4 #2 | 40 mL CHG, 24 g PEG, 20 mL DI water |
| PP-2a | 2 g pure CHG, 2 g PDL-20, 4 g PEG, 30 mL NMP |
| PP-5 | 4 g pure CHG, 2 g PDL-20, 4 g PEG, 30 mL NMP |
| PEG/CHG #4 #25% | 5 mL CHG, 3 g PEG, 25 mL DI water |
| PEG/CHG #6 | 20 mL CHG, 12 g PEG, 12 mL DI water |
| PEG/CHG #7 | 20 mL CHG, 12 g PEG, 15 mL DI water |
| PEG/CHG #8 | 20 mL CHG, 12 g PEG, 18 mL DI water |
| PEG/CHG #9 | 20 mL CHG, 12 g PEG, 20 mL DI water |

Two approaches were explored to find a potential solution to the coating issue. The approach involved experimenting with a secondary coating that was applied to be a protective layer on top of the underlying chlorhexidine coating, referred to herein as an overcoat. The second approach was to blend the chlorhexidine with another material to attempt to provide protection. The results of each of these sets of experiments are summarized below.

### Overcoats—Coating Compositions and Weights

Table 3 lists the coating name and batch number of each pin coated with at least two different coatings (overcoats), the composition of the solution that each experimentation pin was dipped in, the dip speed and dwell time at which each pin was dipped. Every pin was dipped in an acid pretreatment.

TABLE 3

| | Coating | 1^{st} Dip | 1^{st} Dwell | Coating | 2^{nd} Dip | |
|---|---|---|---|---|---|---|
| Batch | Composition of | Speed | Time | Composition | Speed | 2^{nd} Dwell |
| Number | First Dip | (mm/min) | (seconds) | of Second Dip | (mm/min) | Time (sec) |
| M-4-6-20-19 | CHG | 300 | 5 | PEG/CHG | 300 | 5 |
| M-4-6-20-20 | CHG | 300 | 5 | PEG/CHG | 300 | 5 |
| M-4-6-20-21 | CHG | 300 | 5 | PEG/CHG | 300 | 5 |
| M-4-13-20-1 | CHG | 300 | 5 | PEG/CHG #4 | 100 | 5 |
| M-4-13-20-2 | CHG | 300 | 5 | PEG/CHG #4 | 100 | 5 |
| M-4-13-20-3 | CHG | 300 | 5 | PEG/CHG #4 | 100 | 5 |
| M-4-13-20-4 | 0.001% Sodium | 300 | 5 | CHG | 300 | 5 |
| | Alginate | | | | | |
| M-4-13-20-5 | 0.01% Sodium | 300 | 5 | CHG | 300 | 5 |
| | Alginate | | | | | |
| M-4-13-20-6 | 0.1% Sodium | 300 | 5 | CHG | 300 | 5 |
| | Alginate | | | | | |
| M-4-13-20-7 | 1% Sodium | 300 | 5 | CHG | 300 | 5 |
| | Alginate | | | | | |
| M-4-13-20-8 | 10% F127 | 300 | 5 | CHG | 300 | 5 |
| M-4-13-20-9 | FA | 300 | 5 | CHG | 300 | 5 |
| M-4-13-20-10 | FB | 300 | 5 | CHG | 300 | 5 |
| M-4-20-20-1 | CHG | 300 | 5 | PEG/F127 #2 | 100 | 5 |
| M-4-20-20-2 | CHG | 300 | 5 | PEG/F127 #2 | 100 | 5 |
| M-4-20-20-3 | CHG | 300 | 5 | PEG/F127 #2 | 100 | 5 |
| M-4-20-20-4 | CHG | 300 | 5 | 0.1% Alginic | 300 | 5 |
| | | | | acid | | |
| M-4-20-20-5 | CHG | 300 | 5 | FB | 300 | 5 |
| M-4-20-20-6 | 0.1% Alginic | 300 | 5 | CHG | 300 | 60 |
| | Acid | | | | | |
| M-4-30-20-1 | CHG | 300 | 5 | PEG/F127 #1 | 100 | 5 |
| M-4-30-20-2 | CHG | 300 | 5 | PEG/F127 #1 | 100 | 5 |
| M-4-30-20-3 | CHG | 300 | 5 | PEG/F127 #1 | 100 | 5 |
| M-4-30-20-4 | CHG | 300 | 5 | PEG #3g | 100 | 5 |
| M-4-30-20-5 | CHG | 300 | 5 | PEG #3g | 100 | 5 |
| M-4-30-20-6 | CHG | 300 | 5 | PEG #3g | 100 | 5 |
| M-5-11-20-1 | CHG | 300 | 5 | 0.01% CMC | 300 | 5 |
| M-5-11-20-2 | CHG | 300 | 5 | 0.01% CMC | 300 | 5 |
| M-5-11-20-3 | CHG | 300 | 5 | 0.01% CMC | 300 | 5 |
| M-5-11-20-4 | CHG | 300 | 5 | 0.1% CMC | 300 | 5 |
| M-5-11-20-5 | CHG | 300 | 5 | 0.1% CMC | 300 | 5 |
| M-5-11-20-6 | CHG | 300 | 5 | 0.1% CMC | 300 | 5 |
| M-5-18-20-1 | CHG | 300 | 5 | 0.5% CMC | 300 | 5 |
| M-5-18-20-2 | CHG | 300 | 5 | 1% CMC | 300 | 5 |
| M-5-21-20-1 | CHG | 300 | 5 | 1% CMC | 300 | 0 |
| M-5-21-20-2 | CHG | 300 | 5 | 1% CMC | 300 | 0 |
| M-5-21-20-3 | CHG | 300 | 5 | 1% CMC | 300 | 0 |
| M-5-28-20-1* | CMC/CHG #6 | 300 | 5 | CMC/CHG #6 | 300 | 5 |
| M-5-28-20-2* | CMC/CHG #6 | 300 | 5 | CMC/CHG #6 | 300 | 5 |
| M-5-28-20-4 | L-cysteine | 300 | 5 | CHG | 300 | 5 |
| | hydrochloride 1% | | | | | |
| M-5-28-20-5 | L-Glutathione | 300 | 5 | CHG | 300 | 5 |
| | solution 1% | | | | | |
| M-6-11-20-17 | CHG | 300 | 5 | 0.75% CMC | 300 | 0 |
| M-6-11-20-18 | CHG | 300 | 5 | 0.75% CMC | 300 | 0 |
| M-6-11-20-19 | CHG | 300 | 5 | 0.75% CMC | 300 | 0 |
| M-7-6-20-1 | CHG | 300 | 5 | 0.5% CMC | 300 | 0 |
| M-7-6-20-2 | CHG | 300 | 5 | 0.5% CMC | 300 | 0 |
| M-7-6-20-3 | CHG | 300 | 5 | 0.5% CMC | 300 | 0 |
| M-7-9-20-1 | CHG | 300 | 5 | PEG/CHG #4 | 300 | 5 |
| | | | | #25% | | |
| M-7-9-20-2 | CHG | 300 | 5 | PEG/CHG #4 | 300 | 5 |
| | | | | #25% | | |
| M-7-9-20-3 | CHG | 300 | 5 | PEG/CHG #4 | 300 | 5 |
| | | | | #25% | | |

*Dipped 4 times total

### Blends—Coating Compositions and Weights

Table 4 lists the coating name and batch number of each pin coated with a single solution that is mixed with CHG (blend), if the pin was pretreated with acid, the composition of the solution that each experimentation pin was dipped in, and the dip speed and dwell time at which each pin was dipped.

TABLE 4

| | Acid | | Dip | Dwell |
|---|---|---|---|---|
| | Pretreat- | Coating | Speed | Time |
| Batch Number | ment? | Composition | (mm/min) | (seconds) |
| M-3-18-20-1 | — | PEG/CHG #3 | 100 | 5 |
| M-3-18-20-2 | — | PEG/CHG #3 | 100 | 5 |
| M-3-18-20-3 | — | PEG/CHG #3 | 100 | 5 |
| M-3-24-20-7 | Yes | PP-1 | 300 | 5 |
| M-3-24-20-8 | Yes | PP-1 | 300 | 5 |
| M-3-24-20-9 | Yes | PP-1 | 300 | 5 |
| M-3-24-20-10 | Yes | PP-2 | 300 | 5 |
| M-3-24-20-11 | Yes | PP-2 | 300 | 5 |
| M-3-24-20-12 | Yes | PP-2 | 300 | 5 |
| M-3-30-20-13 | — | PEG/CHG #4 | 100 | 5 |
| M-3-30-20-14 | — | PEG/CHG #4 | 100 | 5 |
| M-3-30-20-15 | — | PEG/CHG #4 | 100 | 5 |
| M-5-21-20-7 | Yes | CMC/CHG #3 | 300 | 5 |
| M-5-21-20-8 | Yes | CMC/CHG #4 | 300 | 5 |
| M-5-28-20-3 | Yes | CHG/HPC #1 | 300 | 5 |
| M-6-4-20-1 | Yes | CMC/CHG #5 | 300 | 5 |
| M-6-4-20-2 | Yes | CMC/CHG #7 | 300 | 5 |
| M-6-4-20-3 | Yes | CMC/CHG #8 | 300 | 5 |
| M-6-4-20-4 | Yes | CMC/CHG #9 | 300 | 5 |
| M-6-4-20-5 | Yes | CHG/HPMC #1 | 300 | 5 |
| M-6-22-20-11 | Yes | PP-5 | 300 | 5 |
| M-6-22-20-12 | Yes | PP-5 | 300 | 5 |
| M-6-22-20-13 | Yes | PP-5 | 300 | 5 |
| M-7-27-20-1 | Yes | PEG/CHG #4 | 50 | 5 |
| M-7-28-20-1 | Yes | PEG/CHG #6 | 100 | 5 |
| M-7-28-20-2 | Yes | PEG/CHG #7 | 100 | 5 |
| M-7-28-20-3 | Yes | PEG/CHG #8 | 100 | 5 |
| M-7-28-20-4 | Yes | PEG/CHG #9 | 100 | 5 |

### Non-Wipe Tested Pins

Table 5 displays the experimental pins that were coated but not wipe tested. If a sample either had a poor appearance or an inadequate coating weight, the sample was not wipe tested. The solution name, dip speed, dwell time, and coating weight was listed for each pin.

TABLE 5

| | | | 1^{st} Dip | | | 2^{nd} Dip | |
|---|---|---|---|---|---|---|---|
| | | 1^{st} Dip | Dwell | | 2^{nd} Dip | Dwell | Coating |
| Batch | 1^{st} dip Coating | Speed | Time | 2^{nd} Dip | Speed | Time | Weight |
| Number | Name | (mm/min) | (seconds) | Coating Name | (mm/min) | (seconds) | (μg/cm²) |
| M-4-20-20-7 | 1% alginic acid | 300 | 5 | — | — | — | 190 |
| M-4-30-20-7 | PEG/CHG#4d | 300 | 5 | — | — | — | 125 |
| M-5-26-20-1 | CMC/CHG #6 | 100 | 5 | — | — | — | 0 |
| M-5-26-20-2 | CMC/CHG #6 | 300 | 5 | — | — | — | 9 |
| M-7-9-20-4 | CHG | 300 | 5 | CMC/CHG #5 | 300 | 0 | 277 |
| M-7-9-20-5 | CHG | 300 | 5 | CMC/CHG #7 | 300 | 0 | 287 |
| M-7-9-20-6 | CHG | 300 | 5 | CMC/CHG #9 | 300 | 0 | 297 |
| M-7-20-20-1 | CHG | 300 | 5 | 1% CMC | 300 | 0 | 275 |
| M-7-20-20-2 | CHG | 300 | 5 | 0.75% CMC | 300 | 0 | 235 |
| M-7-20-20-3 | CHG | 300 | 5 | 0.75% CMC | 300 | 0 | 260 |
| M-7-20-20-4 | CHG | 300 | 5 | 1% CMC | 300 | 0 | 253 |
| M-3-23-20-4 | CHG | 300 | 5 | 0.75% CMC | 300 | 0 | 66 |
| M-3-23-20-5 | CHG | 300 | 5 | 0.75% CMC | 300 | 0 | 26 |
| M-3-23-20-6 | CHG | 300 | 5 | 0.75% CMC | 300 | 0 | 47 |
| M-4-6-20-16 | CHG | 300 | 5 | 0.75% CMC | 300 | 0 | −22 |
| M-4-6-20-17 | CHG | 300 | 5 | 0.75% CMC | 300 | 0 | 47 |
| M-4-6-20-18 | CHG | 300 | 5 | 0.75% CMC | 300 | 0 | −16 |
| M-5-21-20-4 | CHG | 300 | 5 | CMC/CHG #7 | 300 | 0 | 91 |
| M-5-21-20-5 | CHG | 300 | 5 | CMC/CHG #7 | 300 | 0 | 60 |
| M-5-21-20-6 | CHG | 300 | 5 | CMC/CHG #7 | 300 | 0 | 69 |
| M-6-22-20-8 | CHG | 300 | 5 | CMC/CHG #7 | 300 | 0 | 70 |
| M-6-22-20-9 | CHG | 300 | 5 | CMC/CHG #7 | 300 | 0 | 74 |
| M-6-22-20-10 | CHG | 300 | 5 | CMC/CHG #7 | 300 | 0 | 80 |

Nails Prepared with Select Glove Resistant Coatings

The most promising coatings were scaled up from pins to nails in order to visualize the effect that water has on the coating. Table 6 displays the coating used, dip speed, and dwell time of each nail.

TABLE 6

| | | | | | | 2^{nd} Dip |
|---|---|---|---|---|---|---|
| | | 1^{st} Dip | 1^{st} Dip Dwell | | 2^{nd} Dip | Dwell |
| | | Speed | Time | 2^{nd} Dip | Speed | Time |
| Batch Number | 1^{st} Dip Coating | (mm/min) | (seconds) | Coating | (mm/min) | (seconds) |
| M-6-18-20-1 | CHG | 300 | 5 | — | — | — |
| M-6-18-20-2 | CHG | 300 | 5 | PEG/CHG #4 | 100 | 5 |
| M-6-18-20-3 | CHG | 300 | 5 | 1% CMC | 300 | 0 |
| M-6-22-20-4 | CHG | 300 | 5 | 0.75% CMC | 300 | 0 |
| M-6-22-20-5 | CHG | 300 | 5 | PEG/F127 #2 | 100 | 5 |
| M-6-22-20-6 | PP-1 | 300 | 5 | — | — | — |
| M-6-22-20-7 | PP-2 | 300 | 5 | — | — | — |
| M-7-13-20-8 | PEG/CHG #4 #2 | 100 | 5 | — | — | — |
| M-7-13-20-9 | PP-5 | 300 | 5 | — | — | — |
| M-8-3-20-10 | PEG/CHG #9 | 100 | 5 | — | — | — |

### Pins Sent for Microbiology and Elution Testing

After viewing the SEM images and coating weights, a decision was made as to which processes/solutions were to be further subjected to microbiology and elution testing. Table 4 above displays the coating name, composition of coating, and dip speed of each batch of pins sent for testing. In each batch of test pins, there were 13 pins dipped. The coated pins were subjected to gamma sterilization before microbiology testing.

In the simplest microbiology test, the samples are placed in a 100% serum containing 5×10⁴ CFU of S. aureus for 24 hours. After this time they are removed, rinsed and placed in DI water. They are then sonicated and vortexed. This solution is then diluted and plated onto agar culture plates. After a 24 hour incubation the number of colonies is counted.

In more challenging microbiology testing, the parts are tested for extended time points, and then analyzed using the same methods for the simpler test.

For the microbiology testing referred to as ‘24 hr rechallenge’, the samples were tested for their ability to resist colonization at 24 hours and they were soaked in the inoculum for 24 hours before being re-challenged by being placed in fresh inoculum for 24 hours. This second time point tests the ability of the coating to resist the microbial challenge on a second day.

For the microbiology testing referred to as ‘48 hr rechallenge’, the samples were tested for their ability to resist colonization at 24 hours and they were soaked in the inoculum for 24 hours before being re-challenged by being placed in fresh inoculum for 24 hours. The pins were then rechallenged again by being placed in fresh inoculum for another 24 hours. This third time point tests the ability of the coating to resist the microbial challenge on a third day.

The elution of the antimicrobial was also measured over time when immersed in 100% serum. All samples were sterilized prior to testing and the break-off tab was removed prior to inserting the pin into the test liquid. The total amount of coating was calculated by taking coating weight in μg/cm² and multiplying it by the nominal surface area of a pin without the break off-tab (5.4 cm²).

All pins sent for testing were titanium, and there were 13 pins prepared in each batch.

From all the blends and overcoat samples made and evaluated, the most promising formulations were selected for additional testing. Formulations were selected if they showed a reduced amount of material lost in the wipe test when compared to a chlorhexidine control and the SEM image showed a consistent coating.

For each of these formulations, a new batch of samples was prepared and subjected to elution and microbiology testing, with one sample retained for SEM analysis. All samples were gamma sterilized prior to testing.

### Overcoats

FIGS. 10-13 show SEM images that represent the four different batches of pins sent for microbiology and elution testing on overcoats.

FIG. 10 is a SEM image of Pin M-5-6-20-13 (coated with CHG+PEG/CHG #4) that represents a batch of pins sent for testing.

FIG. 11 is a SEM image of pin M-6-1-20-13 (coated with CHG+CHG/F127 #2) that represents a batch of pins sent for testing.

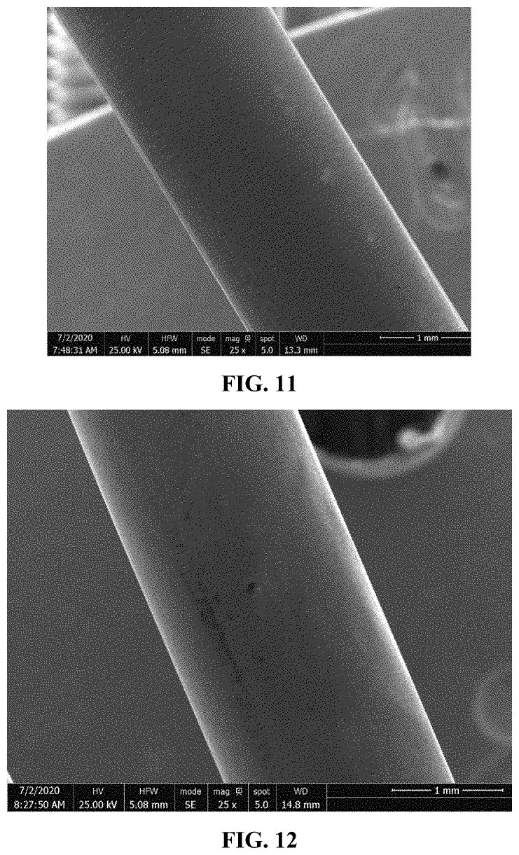

FIG. 12 is a SEM image of pin M-6-8-20-13 (coated with CHG+1% CMC) that represents a batch of pins sent for testing.

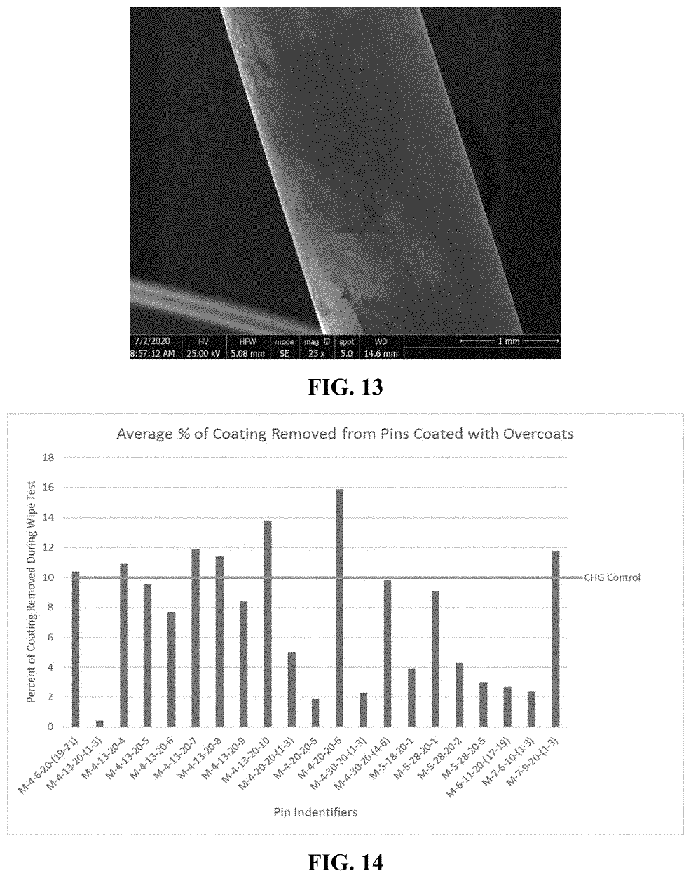

FIG. 13 is a SEM image of pin M-6-11-20-13 (coated with CHG+0.75% CMC) that represents a batch of pins sent for testing.

FIG. 14 is a bar graph illustrating the average percentage of coating removed from pins coated with overcoats (see Table 3 for coating formulations). FIG. 14 displays the amount of coating removed during the wipe test from pins dipped with an overcoat. The horizontal line shows the average amount removed from CHG control pins. Ten of the overcoats had either a higher percentage or less than 1% difference in coating removed compared to the CHG control.

Table 7 shows the average elution results from day 1, 2, and 7 of pins 10-12 from batches prepared with overcoats M-5-4-20, M-6-1-20, M-6-8-20, and M-6-11-20. Batch M-5-6-20 had one pin that eluted 90% of the CHG on the first day. The other two pins in the batch had trace amounts of elution on the second day (approximately 17 μg), but only reached a total of slightly over 70% of coating eluted. Batch M-6-1-20 had about 62% of CHG eluted. Batches M-6-8-20 and M-6-11-20 were both overcoated with CMC dilutions, and both had approximately 85% of CHG eluted from the surface of each pin.

TABLE 7

| | | | | | Total | Coating | |
|---|---|---|---|---|---|---|---|
| | | Day 1 | Day 2 | Day 7 | Eluted | Total | % |
| Description | Pin Batch | μg | μg | μg | μg | μg | Eluted |
| CHG + (PEG/CHG #4) | M-5-6-20-10 | 3992 | — | — | 3992 | 4458 | 90% |
| CHG + (PEG/CHG #4) | M-5-6-20-11 | 3206 | 18.05 | — | 3224 | 4413 | 73% |
| CHG + (PEG/CHG #4) | M-5-6-20-12 | 3128 | 16.35 | — | 3144 | 4489 | 70% |
| CHG/(CHG + F127 #2) | M-6-1-20-10 | 1021 | — | — | 1021 | 1679 | 61% |
| CHG/(CHG + F127 #2) | M-6-1-20-11 | 1052 | — | — | 1052 | 1672 | 63% |
| CHG/(CHG + F127 #2) | M-6-1-20-12 | 1032 | — | — | 1032 | 1657 | 62% |
| CHG/1% CMC | M-6-8-20-10 | 951 | — | — | 951 | 1080 | 88% |
| CHG/1% CMC | M-6-8-20-11 | 893 | — | — | 893 | 1080 | 83% |
| CHG/1% CMC | M-6-8-20-12 | 950 | — | — | 950 | 1080 | 88% |
| CHG/0.75% CMC | M-6-11-20-10 | 933 | — | — | 933 | 1080 | 86% |
| CHG/0.75% CMC | M-6-11-20-11 | 915 | — | — | 915 | 1080 | 85% |
| CHG/0.75% CMC | M-6-11-20-12 | 893 | — | — | 893 | 1080 | 83% |

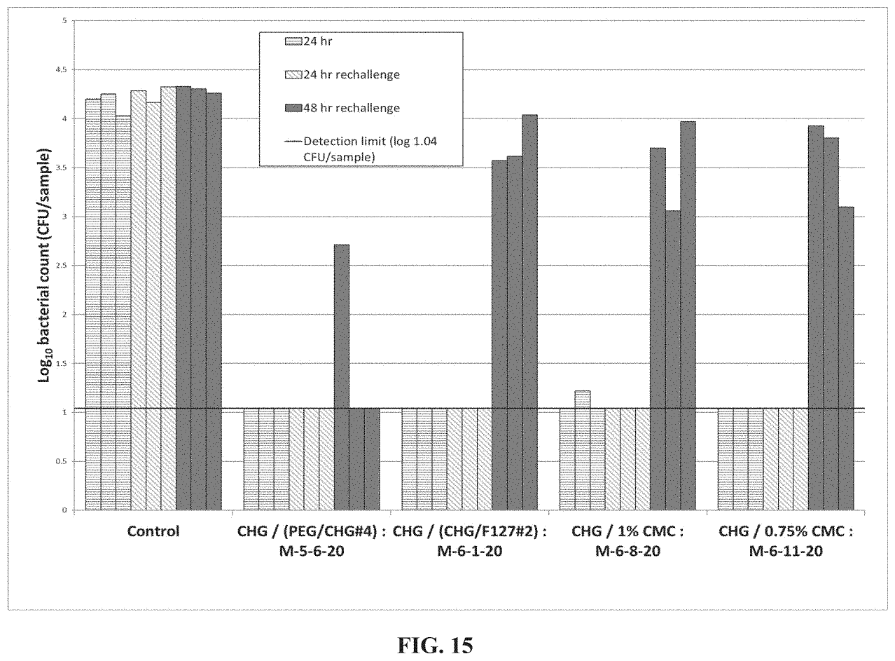

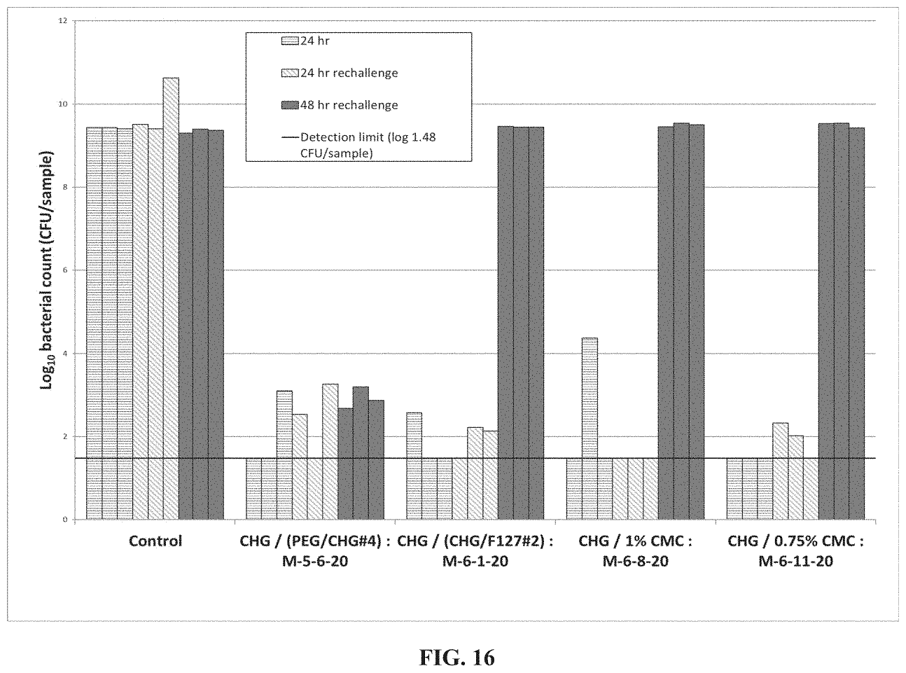

The results after incubating the pins from batches M-5-6-20, M-6-1-20, M-6-8-20, and M-6-11-20 in a solution containing 10⁴ CFU of S. aureus ATCC 25923 are shown in FIGS. 15 and 16.

The results for the surface test show protection of the surface from bacterial colonization for both the first 24 hours and the 24 hours rechallenge for all four variations, except the CHG/1% CMC batch had one pin slightly above detection level during the first 24 hours. For the surface counts, the measurement of the adhered microbes on the coated samples during the 48 hour rechallenge are only slightly lower than the uncoated controls for batches M-6-1-20, M-6-8-20, and M-6-11-20. Batch M-5-6-20 had two pins with microbes at only the detection limit, and one pin that obtained only about half of the microbes as the uncoated control. The overcoats prepared in batches M-5-6-20, M-6-1-20, M-6-8-20, and M-6-11-20 prove to prevent bacteria colonization for the first 48 hours, but then the bacteria will be able to colonize again. This is most likely due to the CHG coating completely eluting the first two days in the solution. The CHG/(PEG/CHG #4) applied to batch M-5-6-20 proved to prevent bacteria colonization for 72 hours, so it is possible that the coating caused a delay in the release of CHG.

FIG. 15 shows microbe counts for the surface for the control and titanium pins coated with overcoats.

The results for the solution test after 24 hours show protection of the solution from colonization in all three pins tested for batch M-6-11-20. M-5-6-20, M-6-1-20, and M-6-8-20 each had one pin whose solution contained microbes after the first 24 hours test, but each of these solutions measured less than the average amount of microbes present for the uncoated control pins. In the analysis of the solution, there is a significant reduction in the number of microbes in the solution when compared to the control for the pin in each batch that allowed microbes. After the 24 hours rechallange, M-5-6-20, M-6-1-20, and M-6-11-20 showed microbes slightly above the detection limit on two pins. M-6-8-20 was the only variation the showed microbes only at the detection level for all three pins during the 24 hours rechallenge. For the solution counts, the measurement of the adhered microbes on the coated samples during the 48 hour rechallenge are about the same as the uncoated controls for batches M-6-1-20, M-6-8-20, and M-6-11-20. Batch M-5-6-20 showed microbes only slightly above the detection limit for all three pins during the 48 hour rechallenge.

FIG. 16 shows microbe counts for the solution for the control and titanium pins coated with overcoats.

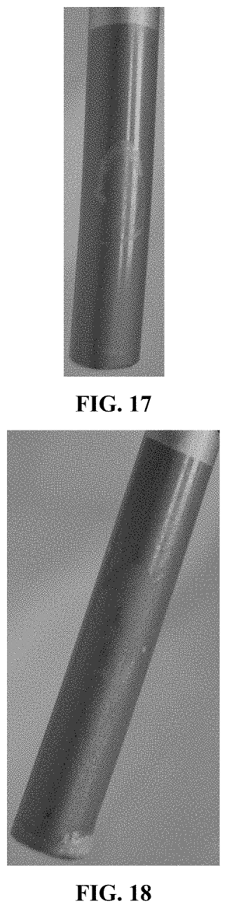

FIGS. 17-21 illustrate the effects that the wet glove test has on each overcoat. After each nail was coated and dried, a gloved thumb was wet with DI water then pressed to the nail. The nail was then dried and a photograph was taken to show the effect of the wet glove test. FIG. 10 shows the CHG control coating in order to compare the various overcoats to the control.

FIG. 17 is a photograph of the aftermath of the wet glove test performed on a nail coated with CHG (M-6-18-20-1).

FIG. 18 is a photograph of the aftermath of the wet glove test performed on a nail coated with the overcoat CHG+PEG/CHG #4 (M-6-18-20-2).

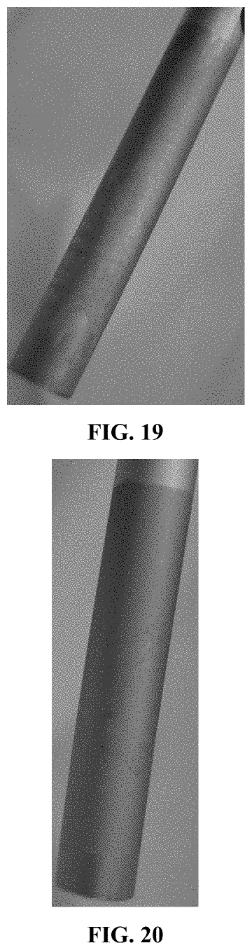

FIG. 19 is a photograph of the aftermath of the wet glove test performed on a nail coated with the overcoat CHG+1% CMC (M-6-18-20-3).

FIG. 20 is a photograph of the aftermath of the wet glove test performed on a nail coated with the overcoat CHG+0.75% CMC (M-6-22-20-4).

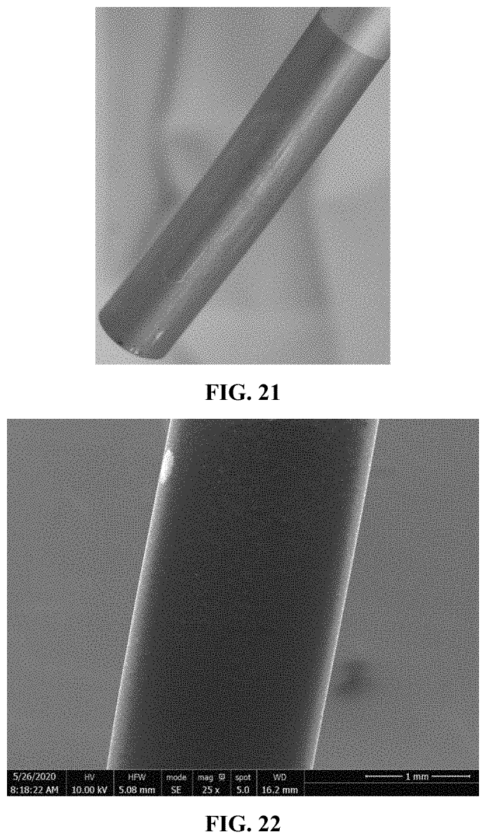

FIG. 21 is a photograph of the aftermath of the wet glove test performed on a nail coated with the overcoat CHG+PEG/F127 #2 (M-6-22-20-5).

### Blended Coatings

FIGS. 22-25 show the SEM images of pins that represent the four different batches of pins sent for microbiology and elution testing on blended coatings.

FIG. 22 is a SEM image of Pin M-5-4-20-13 (coated with PP-1) that represents a batch of pins sent for testing.

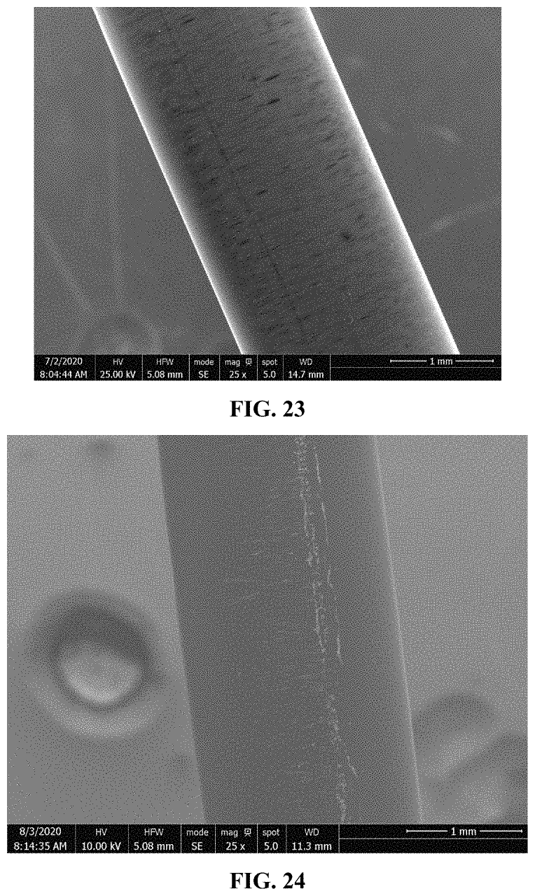

FIG. 23 is a SEM image of pin M-6-2-20-13 (coated with PP-2) that represents a batch of pins sent for testing.

FIG. 24 is a SEM image of pin M-6-29-20-13 (coated with PP-5) that represents a batch of pins sent for testing.

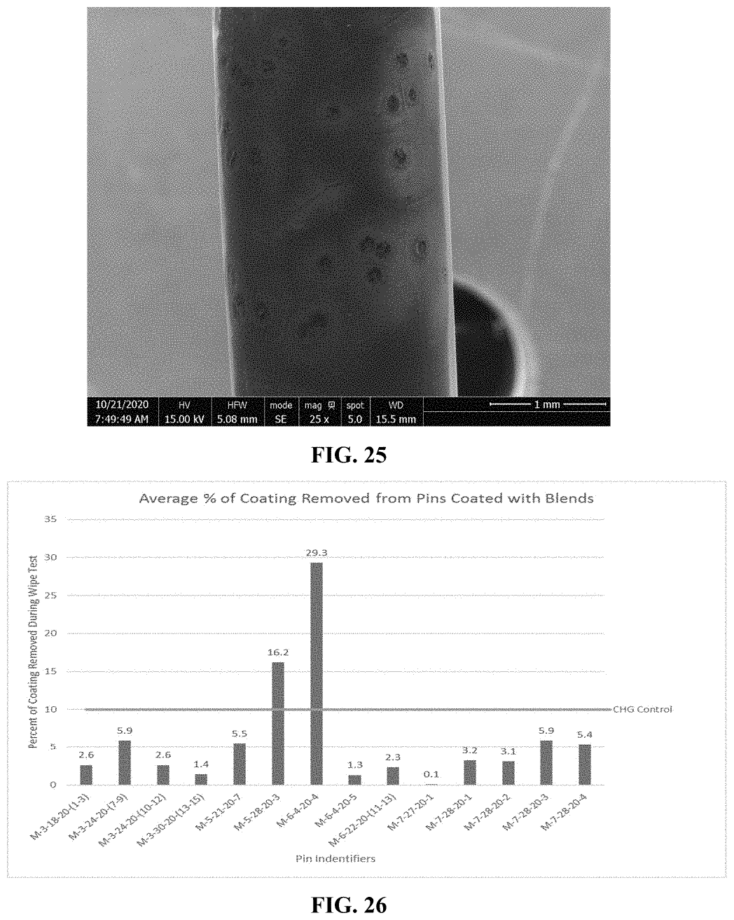

FIG. 25 is a SEM image of pin M-8-4-20-13 (coated with PEG/CHG #9) that represents a batch of pins sent for testing.

FIG. 26 is a bar graph illustrating the average percentage of coating removed from pins coated with blends (see Table 4 for coating formulations). FIG. 26 displays the amount of coating removed during the wipe test from blend-coated pins. The horizontal line shows the average amount removed from CHG control pins. Only two blends had a higher percentage of coating removed than the CHG control. The aim of this research is to develop a coating that is more resistant to water than the CHG coating.

Table 8 shows the average elution results from day 1 of pins 10-12 from batches M-5-4-20, M-6-2-20, M-6-29-20, M-8-4-20. Batch M-5-4-20 only had about 9% of CHG eluted from the surface of the pin. Batch M-6-2-20 eluted about 4% of CHG, and batch M-6-29-20 eluted approximately 7% of CHG. The ultimate destination of the CHG is unclear since less than 10% of the CHG eluted during the testing on pins coated with variants of PP. Batch M-8-4-20 eluted approximately 24% of CHG on the surface of each pin.

TABLE 8

| | | | | | Total | Coating | |
|---|---|---|---|---|---|---|---|
| | | Day 1 | Day 2 | Day 7 | Eluted | Total | % |
| Description | Pin Batch | μg | μg | μg | μg | μg | Eluted |
| PP1 | M-5-4-20-10 | 53 | — | — | 53 | 587 | 9% |
| PP1 | M-5-4-20-11 | 55 | — | — | 55 | 630 | 9% |
| PP1 | M-5-4-20-12 | 53 | — | — | 53 | 592 | 9% |
| PP2 | M-6-2-20-10 | 21 | — | — | 21 | 667 | 3% |
| PP2 | M-6-2-20-11 | 27 | — | — | 27 | 708 | 4% |
| PP2 | M-6-2-20-12 | 33 | — | — | 33 | 757 | 4% |
| PP5 | M-6-29-20-10 | 56 | — | — | 56 | 753 | 7% |
| PP5 | M-6-29-20-11 | 58 | — | — | 58 | 715 | 8% |
| PP5 | M-6-29-20-12 | 58 | — | — | 58 | 777 | 7% |
| PEG/CHG #9 | M-8-4-20-10 | 1576 | — | — | 1576 | 6607 | 24% |
| PEG/CHG #9 | M-8-4-20-11 | 1509 | — | — | 1509 | 6527 | 23% |
| PEG/CHG #9 | M-8-4-20-12 | 1605 | — | — | 1605 | 6823 | 24% |

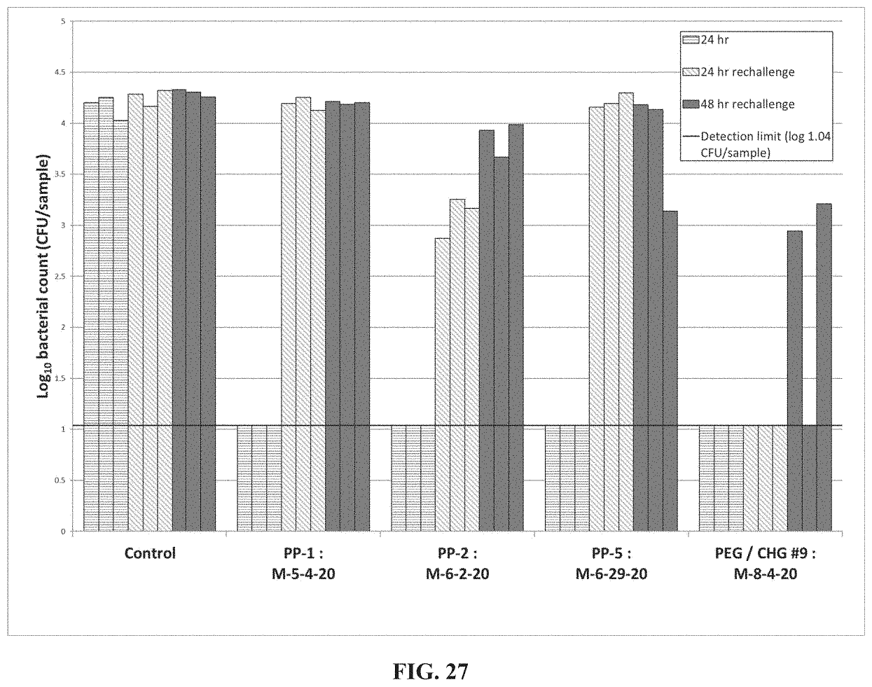

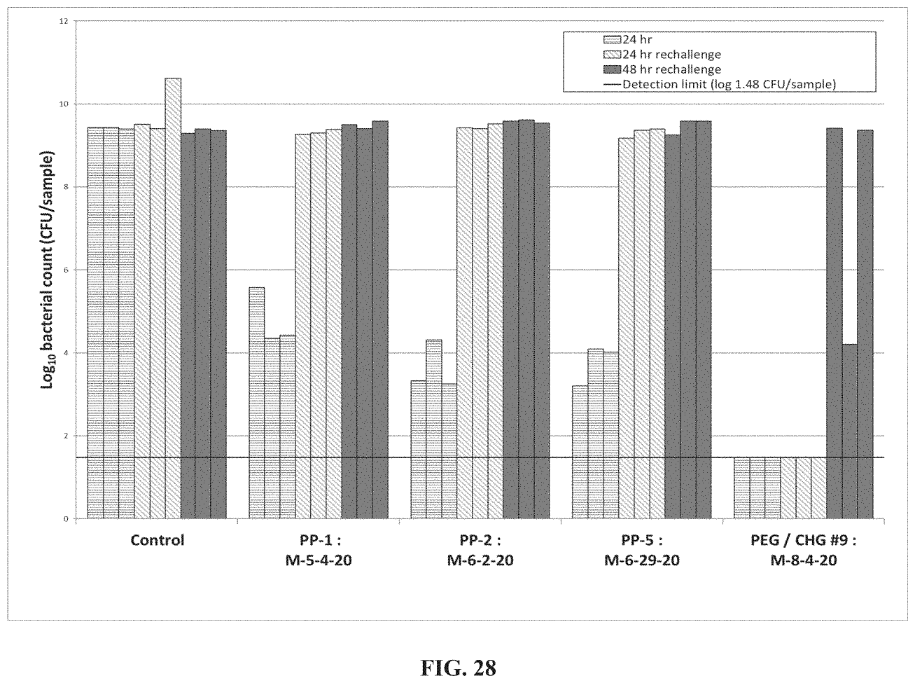

The results after incubating the pins from batches M-5-4-20, M-6-2-20, M-6-29-20, and M-8-4-20 in a solution containing 5×10⁴ CFU of S. aureus ATCC 25923 are shown in FIGS. 27 and 28. The results for the surface test showed protection of the surface from colonization for the first 24 hours. The PEG/CHG #9 blend protected the surface from colonization during the 24 hour rechallenge, and one pin had no microbes above detection level during the 48 hour rechallenge as well.

FIG. 27 shows microbe counts for the surface for the control and titanium pins coated with blends.

For the solution counts of batches M-5-4-20, M-6-2-20, and M-6-29-20, the number of microbes detected was lower than the control pins for the first 24 hours but above the detection limit. The measurement of the adhered microbes on the coated samples during the 24 hour rechallenge and 48 hour rechallenge were about the same as the uncoated controls. The results for the solution test show the PEG/CHG #9 coating with no microbes above the detection limit for the 24 hour and 24 hour rechallenge tests.

FIG. 28 shows microbe counts for the solution for the control and titanium pins coated with blends.

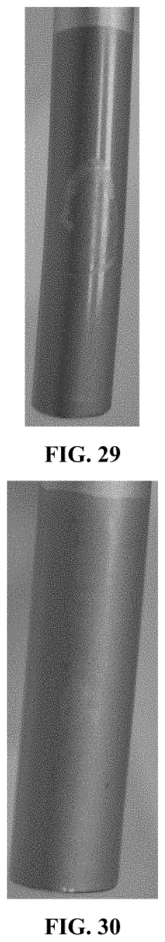

FIGS. 29-34 illustrate the effects that the wet glove test has on each coating. After each nail was coated and dried, a gloved thumb was wet with DI water then pressed to the nail. The nail was then dried and a photograph was taken to show the effect of the wet glove test. FIG. 22 shows the CHG control coating in order to compare the various blended coatings to the control.

FIG. 29 is a photograph of the aftermath of the wet glove test performed on a nail coated with CHG (M-6-18-20-1).

FIG. 30 is a photograph of the aftermath of the wet glove test performed on a nail coated with the blend PP-1 (M-6-22-20-6).

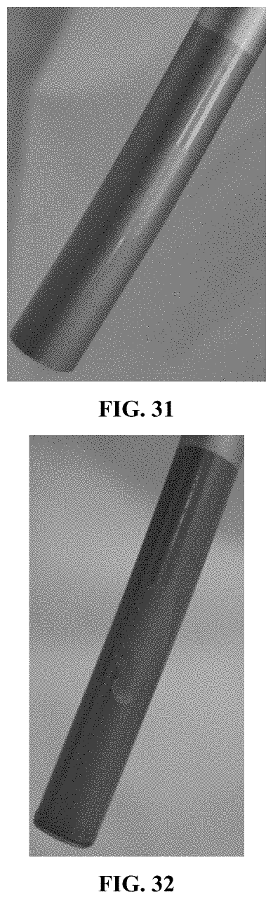

FIG. 31 is a photograph of the aftermath of the wet glove test performed on a nail coated with the blend PP-2 (M-6-22-20-7).

FIG. 32 is a photograph of the aftermath of the wet glove test performed on a nail coated with the blend PEG/CHG #4 #2 (M-7-13-20-8).

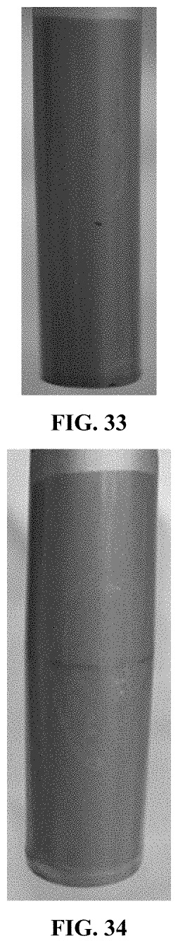

FIG. 33 is a photograph of the aftermath of the wet glove test performed on a nail coated with the blend PP-5 (M-7-13-20-9).

FIG. 34 is a photograph of the aftermath of the wet glove test performed on a nail coated with the blend PEG/CHG #9 (M-8-3-20-10).

After conducting the preceding testing, it was concluded that the pins dipped in CHG/(PEG/CHG #4), CHG/(0.75% CMC), and PEG/CHG #9 have optimum coating consistencies when viewing on the SEM and IM nails, and evaluating microbiology results.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments, or configurations.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

1. An orthopedic implant that comprises a metal substrate and a surface coating comprising chlorhexidine or a salt of chlorhexidine on a surface of the metal substrate.

2. The orthopedic implant of claim 1, wherein the surface coating is a substantially pure coating of chlorhexidine or a salt of chlorhexidine.

3. The orthopedic implant of claim 1, wherein the surface coating further comprises a hydrophilic polymer.

4. The orthopedic implant of claim 3, wherein the hydrophilic polymer is selected from homopolymers and copolymers of alkylene oxides, modified celluloses, and polyvinyl alcohol.

5. The orthopedic implant of claim 1, wherein the surface coating further comprises a hydrophilic polymer and a biodegradable polyester.

6. The orthopedic implant of claim 1, wherein the orthopedic implant further comprises an additional coating comprising a hydrophilic polymer disposed over the surface coating, and wherein the additional coating is different in composition from that of the surface coating.

7. The orthopedic implant of claim 6, wherein the additional coating further comprises chlorhexidine or a salt of chlorhexidine.

8. The orthopedic implant of claim 1, wherein the metal substrate may be formed stainless steels, titanium, titanium alloys, cobalt chromium alloys, zirconium, oxidized zirconium, zirconium alloys, niobium, niobium alloys, hafnium, hafnium alloys, tantalum, tantalum alloys, or nickel-chromium alloys.

9. The orthopedic implant of claim 1, wherein the orthopedic implant is selected from an orthopedic joint implant, a spinal implant, an intramedullary rod, an intramedullary stem, an orthopedic nail, an orthopedic bone plate, or an orthopedic fastener.

10. The orthopedic implant of claim 1, wherein the salt of chlorhexidine may be selected from chlorhexidine digluconate, chlorhexidine diacetate, chlorhexidine dihydrochloride, chlorhexidine phosphanilate, or chlorhexidine dinalidixate.

11. The orthopedic implant of claim 1, wherein the chlorhexidine or salt of chlorhexidine may be present on the metal substrate in an amount ranging from 0.025 μg/cm² to 1000 μg/cm².

12. The orthopedic implant of claim 1, wherein the orthopedic implant is an intramedullary nail and the chlorhexidine or salt of chlorhexidine is present on the metal substrate in a surface concentration ranging from 150 to 300 μg/cm².

13. The orthopedic implant of claim 1, wherein the orthopedic implant is a knee or hip implant and the chlorhexidine or salt of chlorhexidine is present on the metal substrate in a surface concentration ranging from 0.1 to 100 ug/cm² μg/cm².

14. A method of forming an orthopedic implant comprising (a) pretreating a metal substrate to form a pretreated metal substrate and (b) applying a surface coating comprising chlorhexidine or a salt of chlorhexidine on a surface of the pretreated metal substrate to form a surface-coated metal substrate.

15. The method of claim 14, wherein the metal substrate is pretreated with an acidic or basic solution.

16. The method of claim 14, wherein the metal substrate is pretreated with a phosphoric acid solution or a disodium phosphate salt solution.

17. The method of claim 14, the metal surface is pretreated by a surface texturing method selected from chemical etching, electro-etching, anodizing, grit blasting, and cold spray coating processes.

18. The method of claim 14, wherein the surface coating is applied to the pretreated metal substrate by contacting the pretreated metal substrate with a surface coating solution comprising the chlorhexidine or a salt of chlorhexidine.

19. The method of claim 18, wherein the surface coating is applied to the pretreated metal substrate by dip coating the pretreated metal substrate in the surface coating solution.

20. The method of claim 18, further comprising contacting the surface-coated metal substrate with an additional coating solution that comprises a hydrophilic polymer.
